(19) United States
(12) Patent Application Publication
(10) Pub. No.: US 2025/0065141 A1
(43) Pub. Date: Feb. 27, 2025

(54) MAGNETIC FIELD TREATMENT MACHINE

(71) Applicant: MR INC., Ansan-si, Gyeonggi-do (KR)

(72) Inventors: Kyoung Sug SONG, Ansan-si, Gyeonggi-do (KR); Se Un KIM, Ansan-si, Gyeonggi-do (KR)

(21) Appl. No.: 18/852,472

(22) PCT Filed: Apr. 13, 2022
(86) PCT No.: PCT/KR2022/005323

§ 371 Date: Sep. 29, 2024

(30) Foreign Application Priority Data
Apr. 8, 2022 (KR) 10-2022-0043983

(51) Int. Cl. A61N 2/02; H01F 7/20; H01F 27/28

(52) U.S. Cl. CPC A61N 2/02; H01F 7/20; H01F 27/28

(57) ABSTRACT

The present invention relates to a magnetic field treatment device, and provides a magnetic field treatment machine comprising a coil probe for providing multiple stimulation patterns by means of a magnetic field, wherein the coil prove comprises: a coil unit having a plurality of wound coils; and a case including a cover portion, which accommodates the coil unit, and a withdrawal portion, which extends from the cover portion and accommodates withdrawn coils withdrawn from the respective coils of the coil unit.

### TECHNICAL FIELD

The present disclosure relates to a magnetic field treatment device and, more specifically, to a magnetic field treatment device that stimulates or treats a body by generating a magnetic field and intensively irradiating the magnetic field to a portion of the body.

### BACKGROUND ART

In general, magnetic field treatment devices are used to treat or improve various diseases of patients by irradiating a strong magnetic field to a body.

Such magnetic field treatment devices, which are devices that are used for treatment of body parts using a magnetic field, are devices using the principle that a magnetic field is induced by applying a pulsed current to a coil and the magnetic field generated at the coil acts inside a body to generate a bioelectric current, which brings electrophysiological effects in each tissue of the human body.

Magnetic field treatment devices are composed of a power supply that generates a pulsed current, a controller for controlling the power supply, and an electromagnetic coil that generates a magnetic field using the generated pulsed current. Accordingly, the frequency and intensity of a pulse current that is applied to a coil are adjusted in use to reflect the use conditions of treatment devices in accordance with the kinds of diseases or the locations of affected areas.

In particular, when a pulse current of a single frequency is used, patients are quickly adapted to stimulation; but in this case, treatment using a magnetic field is not effectively continued, so the treatment time is increased.

Further, when stimulation of the same pattern is continuously applied to the nerves or muscles of a human body as described above, there is a tendency to become desensitized to stimulation through adaptation.

Further, even when there is a problem with nerves or the function of peripheral sensory nerves is impaired due to diseases, etc., users become more desensitized to continuous stimulation than normal people, so they require stronger stimulation, but strong stimulation may lead to muscle fatigue or tissue damage.

In consideration of these issues, existing stimulators using an electromagnetic field solve such sensory problem (desensitization) by periodically changing the output intensity or the output frequency, but they also repeat stimulation of similar patterns, so the effect is negligible. Further, when the frequency of a pulse current is changed, patients may feel stimulation pain, and when the frequency is greatly changed, the stimulation pain that patients feel increases, which becomes a source of dissatisfaction.

Further, it is required to a greater pulsed current through a power supply in order to generate stronger stimulation, which requires a power supply with high output specifications, making it difficult to reduce the size of stimulation treatment devices and the cost of the devices.

### CITATION LIST

### Patent Literature

### Patent Literature 1

Korean Patent Application Publication No. 10-2009-0063618

### SUMMARY OF INVENTION

### Technical Problem

Accordingly, the present disclosure has been made in an effort to solve the problems of the related art described above, and an objective of the present disclosure is to provide a magnetic field treatment device that simultaneously generates two or more stimulation patterns in a single or a plurality of electromagnetic coils, and thus can efficiently stimulate nerves and muscles, that can generate and supply intensive stimulation using a power supply with low output, and that can be manufactured in a small size.

### Solution to Problem

In order to achieve the objectives described above, a magnetic field treatment device according to the present disclosure is a magnetic field treatment device comprising a single or a plurality of coil probes providing multiple stimulation patterns by means of a magnetic field, in which the coil probe includes: a coil unit having a plurality of wound coils; and a case including a cover portion which accommodates the coil unit and a withdrawal portion which extends from the cover portion and accommodates withdrawn coils withdrawn from the respective coils of the coil unit.

Further, a coupling boss in which a center of the wound coil unit is fitted may be disposed in the case, and supporting ribs surrounding an outer side of the wound coil unit may be disposed on an outer circumference of the coupling boss.

Further, the coupling boss may have a cut portion on a side facing the withdrawal portion of the case to guide withdrawn coils withdrawn from the center of each coil unit to the withdrawal portion.

Further, a heat dissipation member fixing the coil unit inside the case and cooling the coil unit may be disposed in the case.

Further, a heat dissipation fan cooling the coil unit together with the heat dissipation member may be further disposed in the case.

Further, the coil unit may be composed of disc-shaped first and second coils stacked and sequentially wound in a circumferential direction of a plane from the center, the center of the stacked coil may be fitted in the coupling boss, and an outer circumference thereof may be in contact with inner circumferential surfaces of the supporting ribs.

As another example, the coil unit may include a disc-shaped first coil sequentially wound from the center in a circumferential direction of a plane and a disc-shaped second coil sequentially wound from an outer circumference of the first coil in the circumferential direction of the plane, and the center of the first coil may be fitted and fixed in the coupling boss and an outer circumference of the second coil may be in contact with inner circumferential surfaces of the supporting ribs.

As another example, the coil unit may be composed of conical or cylindrical first and second coils stacked and sequentially wound upward from a bottom, the center of the stacked coil unit may be fitted in the coupling boss, and an outer circumference thereof may be in contact with inner circumferential surfaces of the supporting ribs.

### Advantageous Effects of Invention

According to the magnetic field treatment device of the present disclosure, the magnetic field treatment device generates two or more stimulation patterns at a single electromagnetic coil to efficiently stimulate nerves and muscles, whereby it is possible to solve desensitization to stimulation. Further, since intensive stimulation is generated and provided using a power supply of low output, it is possible to reduce the size of the device and decrease the manufacturing cost.

### BRIEF DESCRIPTION OF DRAWINGS

FIG. 1 is a view showing the external appearance of a magnetic field treatment device according to the present disclosure.

FIG. 2 is an exploded view of the magnetic field treatment device according to the present disclosure.

FIG. 3 is an exploded view with a coil unit coupled to a case of the magnetic field treatment device according to the present disclosure.

FIG. 4 is a view showing the configuration of a coil unit according to a first embodiment of the present disclosure.

FIG. 5 is a view showing the configuration of a coil unit according to a second embodiment of the present disclosure.

FIG. 6 and FIG. 7 are views respectively showing examples of output stimulation patterns at the coil unit according to the second embodiment of the present disclosure.

FIG. 8 is a view showing the profile of magnetic field distribution shown at the coil unit according to the second embodiment of the present disclosure and a single coil.

FIG. 9 and FIG. 10 are views showing the configuration of coil units according to third and fourth embodiments of the present disclosure.

FIG. 11 and FIG. 12 are views showing the profiles of magnetic field distribution shown at the coil units according to the third and fourth embodiments of the present disclosure and single units with a shape corresponding to the coil units.

### DESCRIPTION OF EMBODIMENTS

Hereinafter, exemplary embodiments of the present invention are described in detail with reference to the accompanying drawings.

Terminologies used in the present invention are those defined in consideration of functions in the present invention and may depend on users, the intention of an operator, or customs, so the definition of the terminologies should be construed as meanings and concepts corresponding to the technical matters of the present invention.

Further, embodiments of the present disclosure are only examples of the components described in claims without limiting the scope of rights of the present disclosure and are embodiments included in the spirit described throughout the description and including components that can replace the components described in the claims as equivalents.

Further, selective terms used in the following embodiments are used to distinguish one component from other components, and components are not limited to the terms.

In the following description, well-known relevant technologies that may unnecessarily make the spirit of the present disclosure unclear are not described.

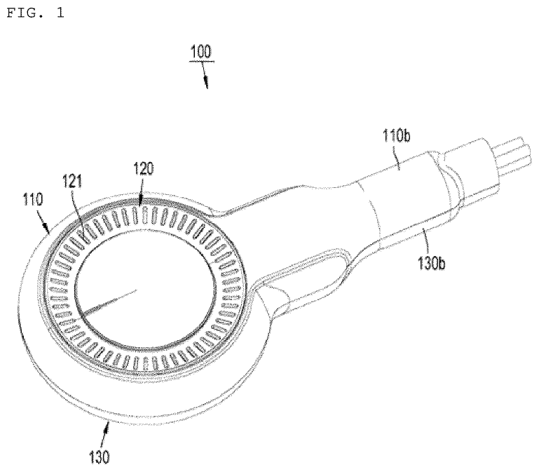

FIGS. 1 to 12 of the accompanying drawings are figures showing examples of a magnetic field generation unit of the magnetic field treatment device according to the present disclosure and a coil unit provided therein, respectively.

A magnetic field treatment device according to the present disclosure, as shown in FIG. 1 to FIG. 3, includes a body (not shown), and a coil probe 100 that withdraws from the body and provides multiple stimulation patterns by means of a magnetic field.

The body has a power supply for supplying a pulsed current, that is, power and a controller for controlling operation of the power supply.

In other words, electric elements provided to generate a magnetic field in the body such as a power unit, a large-capacity capacitor, a switching unit, or the like may be installed in the power supply, and the controller may be implemented as a control panel provided on the front of the body for a user to control operation of the magnetic field treatment device.

A user can adjust electrical elements such as the intensity, frequency, and time of pulse power related to distribution of the magnetic field applied from the coil probe 100 by the controller and can adjust magnetic elements by operating switches, and if a touch panel is provided at the controller, a user can adjust magnetic factors through a graphic user interface (GUI) provided in the screen of the touch panel.

The coil probe 100 has a magnetic field generation coil therein to provide magnetic field stimulation, which corresponds to the intensity, frequency, and time of pulse power determined by a user through controller, to the surface of the user's body, and the magnetic field generation coil may include at least two or more coils.

Specifically, the coil probe 100, as shown in FIG. 1 to FIG. 3, includes a coil unit 200 having a plurality of coils, and cases 110 and 130 having cover portions 110a and 130a which accommodates the coil unit 200 and withdrawal portions 110b and 130b which extends from the cover portions 110a and 130a in one direction and evenly accommodates withdrawn coils withdrawn from the plurality of coils, respectively.

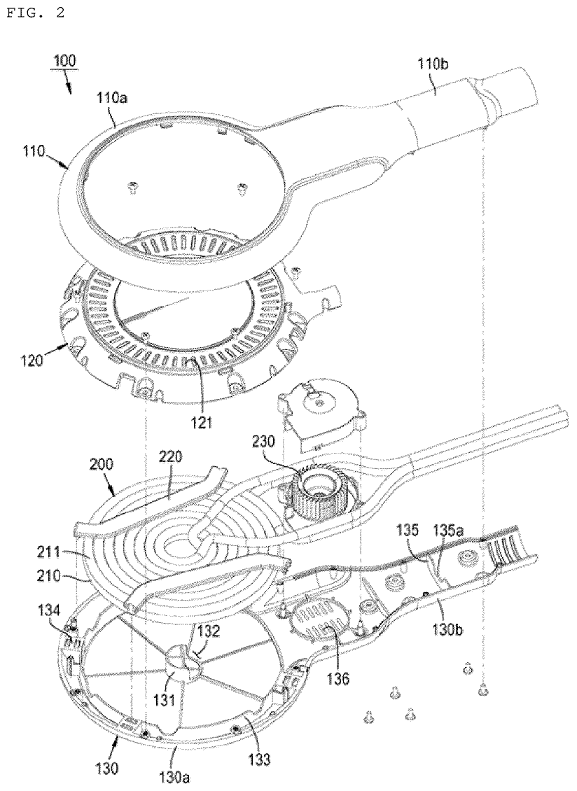

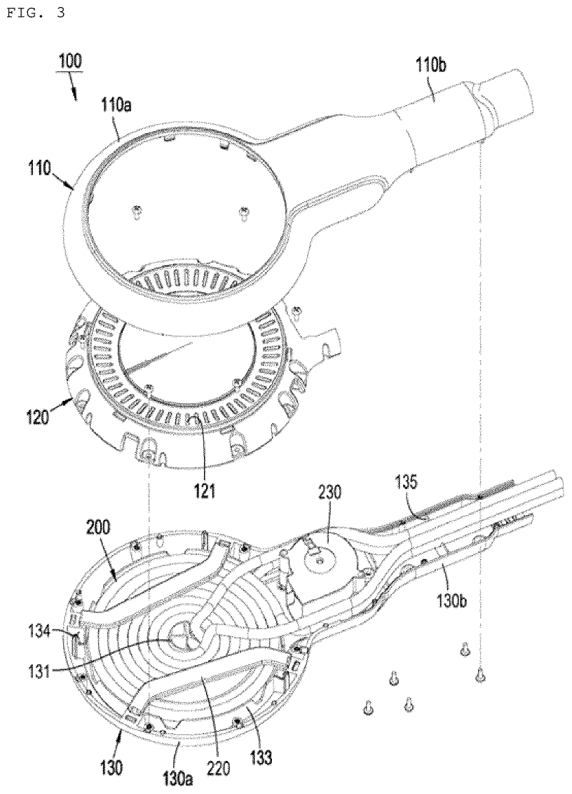

Describing first a case accommodating the coil unit 200, the case, as shown in FIG. 2 and FIG. 3, is formed by separably combining an upper case 110 and a lower case 130. The lower case 130 surrounds and covers the bottom of the coil unit 200 seated therein, and the upper case 110 is combined with the lower case 130 and covers the top of the coil unit 200.

In particular, the upper case 110 and the lower case 130 respectively have the cover portions 110a and 130a which accommodates and covers the coil unit 200, and the withdrawal portions 110b and 130b which accommodates the withdrawn coils withdrawn from the coil unit 200 for supplying power and grounding.

Both the upper and lower cover portions 110a and 130a are each formed in a circular shape corresponding to the winding shape of the coil unit 200 and both the upper and lower withdrawal portions 110b and 130b straightly protrude to correspond to the straight withdrawn coils withdrawn from the coil unit 200.

In this configuration, the cover portion 110a of the upper case 110 may be formed in a ring shape with an open center, and in this case, an intermediate case 120 corresponding to the cover portion 110a of the upper case 110 may be further provided.

The intermediate case 120 is formed in a shape corresponding to the cover portion 110a of the upper case 110 and thread-fastened and fixed to the lower case 130 with the upper case 110 stacked and seated on the intermediate case 120, so the cover portion 110a of the ring-shaped upper case 110 covers the edge of the top of the intermediate case 120.

In this configuration, multiple fastening portions thread-fastened to the lower case 130 are circumferentially formed with regular intervals on the outer circumference of the intermediate case 120. The fastening portions are covered and hidden by the cover portion 110a of the upper case 110.

The upper case 110 having the configuration described above is thread-fastened at the withdrawal portion 110b thereof to the withdrawal portion130b of the lower case 130, thereby retaining the intermediate case 120 between the upper case 110 and the lower case 130.

Further, multiple heat dissipation holes 121 are circumferentially formed like a grill on the top of the intermediate case 120, that is, the region not hidden by the upper case 110. The heat dissipation holes 121 are provided to discharge and dissipate heat generated at the coil unit 200 to be described below to the outside of the coil probe 110.

Meanwhile, a coupling boss 131 in which the center of the wound coil unit 200 is fitted protrudes upward inside the cover portion 130a of the lower case 130, and supporting ribs 133 covering the outer side of the wound coil unit 200 are disposed on the outer circumference of the coupling boss 131.

In particular, the coupling boss 131 has a cut portion 132 on aside facing the withdrawal portion 130b of the lower case 130, and the withdrawn coils withdrawn to the withdrawal portion 130b from the center of the wound coil unit 200 are withdrawn with minimal bending without interference with the coupling boss 131 by the cut portion 132, whereby it is possible to prevent unnecessary deformation of the coil unit 200.

In this configuration, for stable coupling of the coil unit 200 fitted therein, it is preferable that the coupling boss 131 has a height the same as or greater than the height of the coil unit 200, and it is preferable that the outer diameter of the coupling boss 131 is the same as or smaller than the inner diameter of the center portion of the wound coil unit 200.

The supporting ribs 133 are formed like fences protruding upward on the inner edge of the lower case 130 spaced apart from the coupling boss 131 and surround the outer side of the wound coil unit 200, whereby it is possible to prevent loosening of the wound coil unit 200.

Further, fastening blocks 134 to which heat dissipation members 220 to be described below are fastened and fixed are provided at regular intervals between the supporting ribs 133 and the outer circumference of the cover portion 130a.

The withdrawn coils withdrawn from the coil unit 200 for supplying power and grounding are evenly accommodated in both the upper and lower withdrawal portions 110b and 130b protruding and extending from a side of each of both the upper and lower cover portions 110a and 130a.

To this end, a guide plate 135 having a seat groove 135a in which the withdrawn coils are seated and supported is disposed in the upper and lower withdrawal portions 110b and 130b or the lower withdrawal portion 130b. Accordingly, the withdrawn coils are withdrawn while being seated and supported in the seat groove 135a of the guide plate 135, so the coils are evenly arranged and prevented from twisting and the like.

Multiple intake ports136 enabling external air from flowing inside when a heat dissipation pan 230 to be described below is operated are formed like a grill at the front ends of the withdrawal portions 110b and 130b, that is, at the joints connecting to the cover portions 110a and the 130a.

Meanwhile, before describing the coil unit 200, even though a double coil unit is exemplified in this embodiment, it should be noted that triple or more coil unit may be configured.

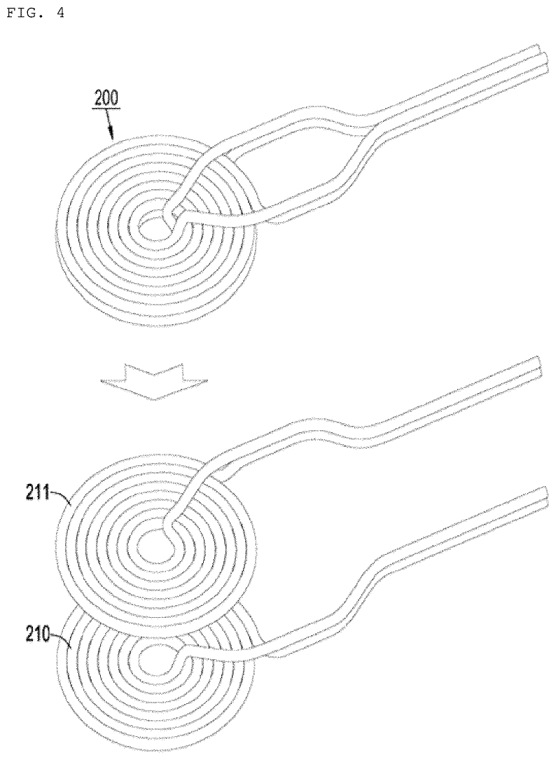

The coil unit 200, as shown in FIG. 4 to FIG. 7, is composed of first and second coils 210 and 211, and the first and second coils 210 and 211 are connected to magnetic field generation circuits (not shown), respectively.

In particular, the coil unit 200 composed of the first and second coils 210 and 211, as shown in FIG. 4 to FIG. 7, may be provided in various structures, but the form shown in FIG. 4 is described as a basic structure.

That is, the basic form of the coil unit 200, as shown in FIG. 4, is composed of disc-shaped first and second coils 210 and 211 stacked and sequentially wound in the circumferential direction of a plane from the center. The center of the stacked coil unit 200 is fitted in the coupling boss 131, and the outer circumference thereof is in contact with the inner circumferential surfaces of the supporting ribs 133.

The first and second coils 210 and 211 constituting the coil unit 200, as described above, are provided in disc shapes wound in a plane to have the same diameter, and the disc-shaped first and second coils 210 and 211 are stacked in two layers. However, in the present disclosure, the coil unit 200 is not limited to the basic form and may be provided in a conical form or a cylindrical form or polygonal forms such as a triangle, a rectangle, or the like.

Meanwhile, the first and second coils 210 and 211 of this embodiment are wound from the inside of the center of the coil unit 200 in a plane in the circumferential direction of the plane such that the diameter is increased, and the inner ends and the outer ends of the first and second coils 210 and 211 are provided as electrode terminals exposed to the outside, respectively. In particular, one electrode terminal of a plurality of electrode terminals withdrawn from a single coil is a terminal for supplying power and the other electrode terminal is a terminal for grounding.

The coil unit 200 having this structure has a structure in which the second coil 211 is stacked on the disc-shaped first coil 210 having the same diameter, a first magnetic field having intensity and a frequency controlled by a separate magnetic field generation circuit (not shown) is formed at the first coil 210, and a second magnetic field having intensity and a frequency controlled by a separate magnetic field generation circuit is formed at the second coil 211, whereby it is possible to simultaneously generate magnetic field stimulation having different intensities and frequencies through one coil probe 100.

In addition, a magnetic field stronger than the magnetic fields generated at each coil is generated at the overlap portion of the stacked first and second coils 210 and 211, and the magnetic field ranges of the first and second coils 210 and 211 have a characteristic of being formed similar to that of a single coil.

Accordingly, since the first and second coils 210 and 211 constituting the coil unit 200 are wound in a disc shape and stacked, the volume of the coil unit 200 is decreased, so the size of the magnetic field treatment device can be reduced.

Hereafter, in the description of the coil unit 200, the coil positioned at the lower portion and inner side of the coil unit 200 is referred to as the first coil 210 and the coil positioned at the upper portion and outer side is referred to as the second coil 211.

Meanwhile, a plurality of heat dissipation members 220 crossing the coil unit 200 is disposed at both sides on the top of the coil unit 200. Both ends of the heat dissipation members 220 are thread-fastened and fixed to the fastening blocks 134 of the case, that is, the lower case 130.

The heat dissipation members 220 are provided as rectangular tube-shaped heat sinks that can maximize the contact area with the coil unit 200, thereby quickly dissipating heat generated at the coil unit 200.

In particular, the heat sinks of the heat dissipation members 220 are made of a non-metallic material that is not affected by the magnetic field generated at the coil unit 200, and the non-metallic heat sinks may be freely changed in design in terms of shape and structure that can quickly dissipate heat.

Further, the heat dissipation members 220 support the top of the coil unit 200 to be in close contact with the inner surface of the lower case 130, thereby being able to maximize a heat transfer area and firmly support the coil unit 200.

In addition, an air cooling-type heat dissipation fan 230 for maximizing the heat dissipation effect for the coil unit 200 together with the heat dissipation members 220 is further provided inside the case, that is, the lower case 130.

The heat dissipation fan 230 is disposed as a sirocco fan, and the center of the fan is positioned at the intake port 136 of the lower case 130 and blades thereof are positioned close to the heat dissipation members 220. Accordingly, the heat dissipation fan 230 that is a sirocco fan suctions external air into the fan through the intake port 136 of the lower case 130 and then blows the external air to the heat dissipation members 220 using the blades on the outer circumference thereof, whereby it is possible to quickly cool the heat dissipation members 220 that have exchanged heat with the coil unit 220.

Meanwhile, the coil unit 200 may be provided in various forms in which the shape and arrangement of the first and second coils 210 and 211 are changed, other than the basic form described above, so only other embodiments of the coil unit 200 are described hereafter.

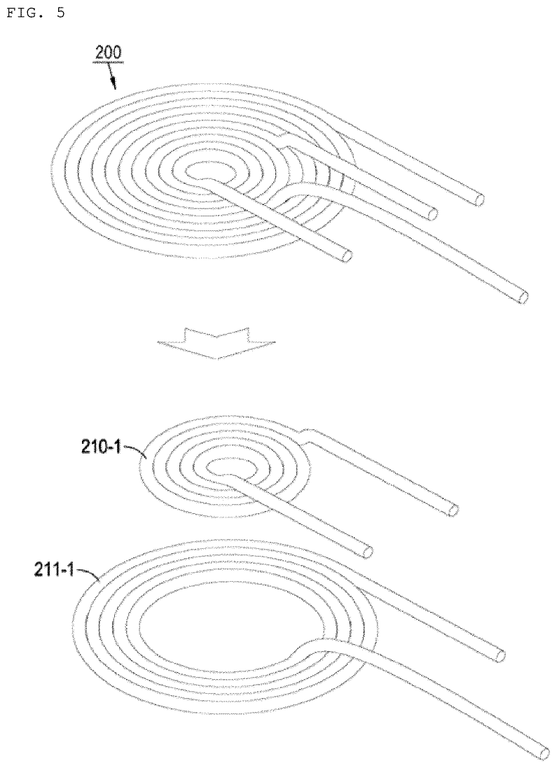

First, in a coil unit 200 according to a second embodiment, as shown in FIG. 5, first and second coils 210-1 and 211-1 that have different diameters are arranged in a plane.

The coil unit 200 of this embodiment, as shown in FIG. 5, includes a disc-shaped first coil 210-1 sequentially wound from the center in the circumferential direction of a plane and a disc-shaped second coil 211-1 sequentially wound from the outer circumference of the first coil 210-1 in the circumferential direction of the plane, in which the center of the first coil 210-1 is fitted and fixed in the coupling boss 131 and the outer circumference of the second coil 211-1 is in contact with the inner surfaces of the supporting ribs 133.

That is, the first coil 210-1 is wound from the inside of the center of the coil unit 211-1 in the circumferential direction of a plane such that the diameter is increased and has a first electrode terminal withdrawn from the inner end thereof and a second electrode terminal withdrawn from the outer end thereof.

Further, similarly, the second coil 211-1 is wound from the outer circumference of the first coil 210-1 in the circumferential direction of a plane such that the diameter is increased and has a first electrode terminal withdrawn from the inner end thereof and a second electrode terminal withdrawn from the outer end thereof.

The first and second coils 210-1 and 211-1 configured as described above are connected to the power unit and the switching unit of the magnetic field generation circuits (not shown), respectively, and can form magnetic fields with controlled intensity and frequencies, respectively. Accordingly, it is possible to simultaneously generate magnetic field stimulation having different intensities and frequencies through a single coil probe 100.

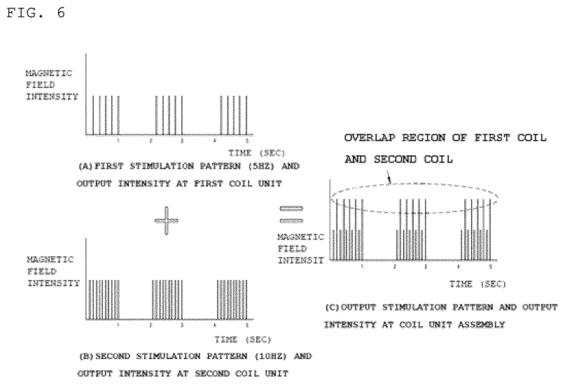

Meanwhile, referring to FIG. 6 for stimulation patterns and output intensity obtained using the coil unit 200 according to the second embodiment, in FIG. 6, (a) shows a first stimulation pattern with first magnetic field intensity and a frequency of 5 Hz, (b) shows a second stimulation pattern with second magnetic field intensity and a frequency of 10 Hz, and (c) shows a stimulation pattern at the coil unit composed of the first and second coils.

As shown in FIG. 6, the first stimulation pattern with a frequency of 5 Hz and intensity of 1 is generated in the first coil unit 210-1, and the second stimulation pattern with a frequency of 10 Hz and intensity of 1 is generated in the second coil 211-1. Accordingly, the stimulation pattern at the coil unit 200 composed of the first and second coils 210-1 and 211-1 is an output stimulation pattern having a combination of the first and second stimulation patterns, and magnetic intensities overlap each other at the interface region of the first coil 210-1 and the second coil 211-1.

That is, the coil unit 200 represents high magnetic field intensity at the physical overlap position of the joint of the first coil 210-1 at the inside and the second coil 211-1 at the outside, and when output of the magnetic fields generated by the coils 210-1 and 211-1 overlap in relation to time, the output is increased by overlap at the point in time. Such an increase of output at the overlap region can generate high output intensity using low power, so it is not required to use a high-power power unit in order to provide high output intensity.

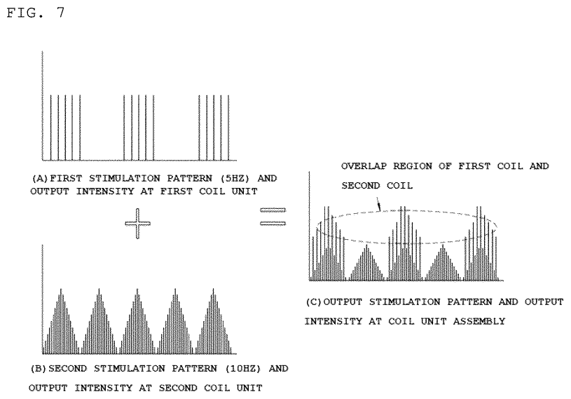

FIG. 7 shows an example of other output stimulation patterns formed by the first coil 210-1 and the second coil 211-1, in which a first stimulation pattern with a frequency of 5 Hz and uniform output intensity is formed at the first coil 210-1, a second stimulation pattern with a frequency of 10 Hz and output intensity that changes over time is formed at the second coil 211-1, so, finally, a new stimulation pattern that is a combination of the first stimulation pattern and the second stimulation pattern is formed.

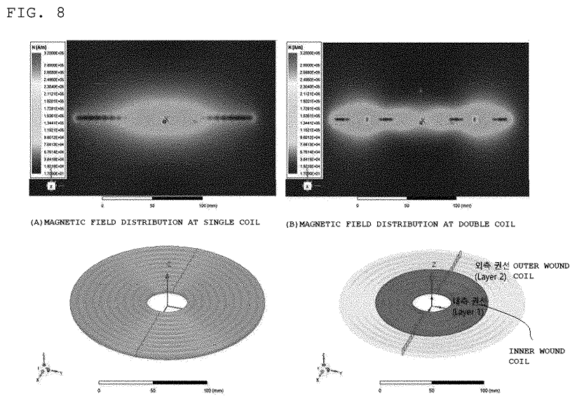

Further, FIG. 8 shows simulations of magnetic field distribution of a common single coil and a coil unit according the present disclosure, in which it can be seen that a stimulation pattern is obtained in a fixed form in accordance with the intensity and frequency of a pulse current that is supplied to the coil in the single coil, but a magnetic field distribution is obtained in various forms at the coil unit 200 of the present disclosure that is a double coil depending on the intensity and frequency of the pulse current that is applied to the first coil 210-1 and the second coil 211-1. For example, when a strong pulse current is applied to the outer coil (second coil 211-1) and a relatively weak pulse current is applied to the inner coil (first coil 210-1), it can be seen that, as shown in FIG. 8, magnetic field distribution with a magnetic field stronger on the outside the coil unit 200 in comparison to the inside is obtained. In the opposite case, magnetic field distribution with a magnetic field stronger on the inside the coil unit in comparison to the outside is obtained.

In addition, in the physical region of the joint between the first coil 210-1 and the second coil 211-1, magnetic fields overlap each other, so high magnetic field intensity is obtained. The overlap magnetic field intensity can also be adjusted by adjusting the first stimulation pattern and the second stimulation pattern, whereby it is possible to provide more various stimulation patterns to skin tissues through the coil probe 100.

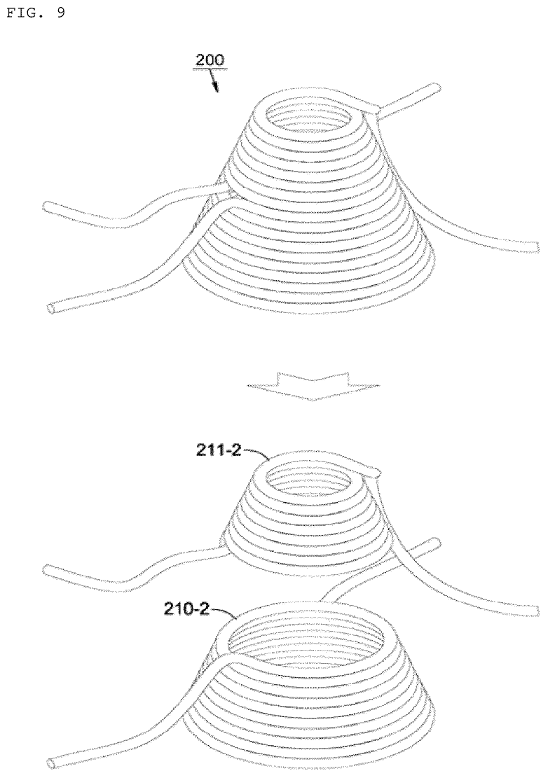

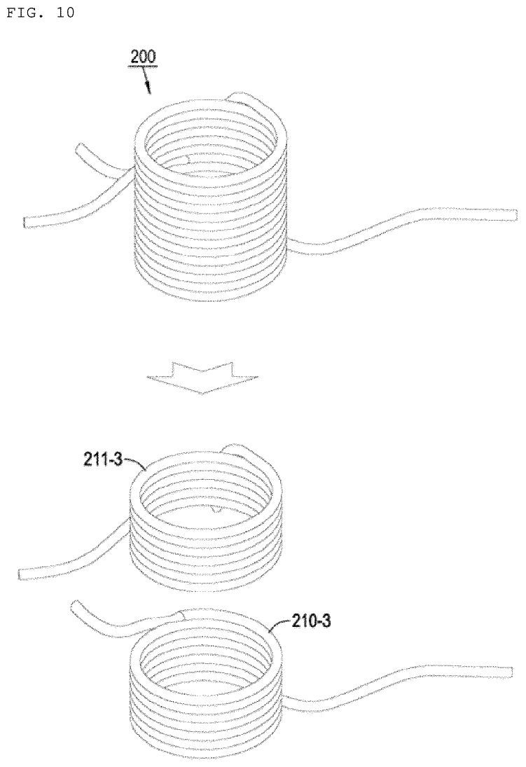

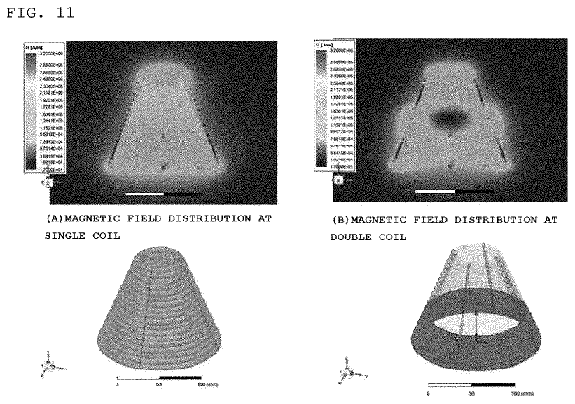

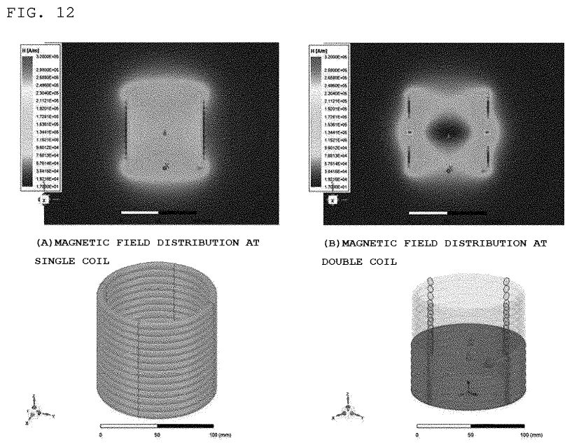

FIG. 9 and FIG. 10 are views showing the configuration of coil units according to third and fourth embodiments that are other embodiments of the present disclosure, and FIG. 11 and FIG. 12 are views showing the profiles of magnetic field distribution shown at the coil units according to the third and fourth embodiments of the present disclosure and single units with a shape corresponding to the coil units.

A coil unit 200 according to the third embodiment, as shown in FIG. 9, has a structure in which first and second coils 210-2 and 211-2 having different diameters are stacked in a conical shape, and a coil unit 200 according to the fourth embodiment, as shown in FIG. 10, has a structure in which first and second coils 210-3 and 211-3 having the same diameter are stacked in a cylindrical shape, so both have a three-dimensional shape.

That is, the coil unit 200 according to the third embodiment, as shown in FIG. 9, includes a conical first coil 210-2 sequentially wound upward from the bottom such that the diameter gradually decreases and a small conical second coil 211-2 sequentially wound upward from the upper end of the first coil 210-2 such that the diameter gradually further decreases, in which the conical first and second coils 210-2 and 211-2 are stacked in two layers.

Further, the coil unit 200 according to the fourth embodiment, as shown in FIG. 10, includes a cylindrical first coil 210-3 sequentially wound upward from the bottom while rotating in place and a cylindrical second coil 211-3 sequentially wound upward from the upper end of the first coil 210-3 while rotating in place, in which the cylindrical first and second coils 210-3 and 211-3 are stacked in two layers.

As described above, the coil unit 200 in which the first and second coils 210-2, 211-2, 210-3, and 211-3 are stacked in a conical or cylindrical shape has a center fitted in the coupling boss 131 and an outer circumference being in contact with the inner surfaces of the supporting ribs 133.

Of course, the coupling boss 131 may be formed and provided in a shape corresponding to the shapes of the coil units of the embodiments.

The coil unit 200 may be formed in three-dimensional shapes such as a polyhedral or polygonal cylindrical shape other than the conical or cylindrical shape.

Accordingly, since the coil units 200 according to these embodiments are also composed of first and second coils 210-2, 211-2, 210-3, and 211-3 similar to the embodiment described above, they provide magnetic distribution in various forms.

That is, as shown in FIG. 11 and FIG. 12, since magnetic intensities overlap each other at the interface region of the first coils 210-2 and 210-3 at the lower portion and the second coil 211-2 and 211-3 at the upper portion, strong magnetic field intensity is generated at the overlap portion of the first and second coils 210-2, 211-2, 210-3, and 211-3, and a magnetic field range similar to that of a single coil is generated.

In particular, the coil units 200 according to the third and fourth embodiments are used for various body parts such as the wrist, ankle, calf, or the like of human, thereby being able to simultaneously provide various multiple stimulation patterns to desired parts of a body.

Further, similar magnetic field intensity and magnetic field ranges are generated at the coil units 200 according to the first embodiment and the fourth embodiment described above.

Although the present disclosure was described in detail with reference to detailed embodiments, the embodiments are provided only to describe the present disclosure in detail and the present disclosure is not limited to the embodiments. Further, it is apparent that the present disclosure may be changed and improved by those skilled in the art without departing from the spirit of the present disclosure.

Simple changes and modifications of the present invention are included in the range of the present invention and the detailed protection range of the present invention will be made clear by the accompanying claims.

### REFERENCE SIGNS LIST

100: Coil probe
110: Upper case
110a: Cover portion
110b: Withdrawal portion
120: Intermediate case
121: Heat dissipation hole
130: Lower case
130a: Cover portion
130b: Withdrawal portion
131: Coupling boss
132: Cut portion
133: Supporting rib
134: Fastening block
135: Guide plate
135a: Seat groove
136: Intake port
200: Coil unit
210, 211, . . . , 210-3, 211-3: First and second coils
220: Heat dissipation member
230: Heat dissipation fan

1. A magnetic field treatment device comprising a single or a plurality of coil probes providing multiple stimulation patterns by means of a magnetic field,
wherein the coil probe comprises:
a coil unit having a plurality of wound coils; and
a case having a cover portion accommodating the coil unit and a withdrawal portion extending from the cover portion and accommodating withdrawn coils withdrawn from the respective coils of the coil unit.

2. The magnetic field treatment device of claim 1, wherein a coupling boss in which a center of the wound coil unit is fitted is disposed in the case, and supporting ribs surrounding an outer side of the wound coil unit are disposed on an outer circumference of the coupling boss.

3. The magnetic field treatment device of claim 2, wherein the coupling boss has a cut portion on a side facing the withdrawal portion of the case and guides withdrawn coils withdrawn from the center of the coil unit to the withdrawal portion.

4. The magnetic field treatment device of claim 1, wherein a heat dissipation member fixing the coil unit inside the case and cooling the coil unit is disposed in the case.

5. The magnetic field treatment device of claim 4, wherein a heat dissipation fan cooling the coil unit together with the heat dissipation member is further disposed in the case.

6. The magnetic field treatment device of claim 2, wherein the coil unit is composed of disc-shaped first and second coils stacked and sequentially wound in a circumferential direction of a plane from the center, the center of the stacked coil unit is fitted in the coupling boss, and an outer circumference thereof is in contact with inner circumferential surfaces of the supporting ribs.

7. The magnetic field treatment device of claim 2, wherein the coil unit includes a disc-shaped first coil sequentially wound from the center in a circumferential direction of a plane and a disc-shaped second coil sequentially wound from an outer circumference of the first coil in the circumferential direction of the plane, and the center of the first coil is fitted and fixed in the coupling boss and an outer circumference of the second coil is in contact with inner surfaces of the supporting ribs.

8. The magnetic field treatment device of claim 2, wherein the coil unit is composed of conical or cylindrical first and second coils stacked and sequentially wound upward from a bottom, the center of the stacked coil unit is fitted in the coupling boss, and an outer circumference thereof is in contact with inner circumferential surfaces of the supporting ribs.
